(19) United States
(12) Patent Application Publication
(10) Pub. No.: US 2026/0220328 A1
(43) Pub. Date: Jul. 30, 2026

(54) METHOD, APPARATUS, AND MEDIUM FOR PREDICTING TENSILE-COMPRESSIVE FATIGUE CHARACTERISTICS OF A RECYCLED ASPHALTMIXTURE

(71) Applicant: FOJIAOKE TIANNUO (GUANGDONG) MATERIALS CO., LTD., FOSHAN (CN)

(72) Inventors: HAO LI, FOSHAN (CN); GUODONG ZENG, FOSHAN (CN); YANG FANG, FOSHAN (CN); HUAJUN MENG, FOSHAN (CN); YONHKANG LIN, FOSHAN (CN); YOUWEI TAN, FOSHAN (CN); HAOLONG YANG, FOSHAN (CN)

(73) Assignee: FOJIAOKE TIANNUO (GUANGDONG) MATERIALS CO., LTD., FOSHAN (CN)

(21) Appl. No.: 19/426,521

(22) Filed: Dec. 19, 2025

Related U.S. Application Data
(63) Continuation of application No. PCT/CN2025/075025, filed on Jan. 25, 2025.

(51) Int. Cl. G06F 30/20; C08L 95/00

(52) U.S. Cl. CPC G06F 30/20; C08L 95/00; C08L 2555/10

(57) ABSTRACT

The present invention discloses a method for predicting the tensile-compressive fatigue characteristics of a recycled asphalt mixture. The method comprises: constructing a target tensile dynamic modulus master curve for a target asphalt mixture; extracting a target tensile effective elasticity ratio from the target tensile dynamic modulus master curve; inputting the target tensile effective elasticity ratio into a pre-established viscoelastic-tensile fatigue curve to generate target tensile fatigue characteristics; constructing a target compressive dynamic modulus master curve for the target asphalt mixture; extracting a target compressive effective elasticity ratio from the target compressive dynamic modulus master curve; and inputting the target compressive effective elasticity ratio into a pre-established first viscoelastic-compressive fatigue curve to generate target compressive fatigue characteristics. By using the present invention, the prediction accuracy of fatigue characteristics can be significantly improved, and the amount and time required for fatigue testing can be reduced.

### TECHNICAL FIELD

The present invention relates to the technical field of analyzing recycled asphalt mixtures, and more particularly, to a method, apparatus, and medium for predicting the tensile-compressive fatigue characteristics of a recycled asphalt mixture.

### BACKGROUND

The extensive application of hot recycled asphalt mixture (HRAM) not only aligns with “dual carbon” objectives but also alleviates shortages of stone materials. However, the selection of an appropriate method for evaluating its fatigue performance has long been a subject of debate.

Currently, researchers primarily predict fatigue performance using test methods such as three-point bending, four-point bending, and indirect tensile fatigue tests. These methods assume that HRAM is an elastic material, whereas asphalt mixture is, in fact, a viscoelastic material. Consequently, results from three-point bending, four-point bending, and indirect tensile fatigue tests often yield conclusions where the fatigue performance rankings of HRAM with different recycled content are inconsistent with established knowledge.

In summary, to clearly and effectively differentiate the fatigue performance of HRAM, there is a need to establish a new fatigue prediction method.

### SUMMARY

The technical problem to be solved by the present invention is to provide a method, apparatus, and medium for predicting the tensile-compressive fatigue characteristics of a recycled asphalt mixture, which can significantly improve the prediction accuracy of fatigue characteristics while reducing the amount and time required for fatigue testing.

To solve the aforementioned technical problem, the present invention provides a method, apparatus, and medium for predicting the tensile-compressive fatigue characteristics of a recycled asphalt mixture, comprising:
constructing a target tensile dynamic modulus master curve for a target asphalt mixture;
extracting a target tensile effective elasticity ratio from the target tensile dynamic modulus master curve;
inputting the target tensile effective elasticity ratio into a pre-established viscoelastic-tensile fatigue curve to generate target tensile fatigue characteristics;
constructing a target compressive dynamic modulus master curve for the target asphalt mixture;
extracting a target compressive effective elasticity ratio from the target compressive dynamic modulus master curve; and
inputting the target compressive effective elasticity ratio into a pre-established first viscoelastic-compressive fatigue curve to generate target compressive fatigue characteristics.

In an improvement of the above embodiment, the target tensile effective elasticity ratio is the ratio of the sum of an elastic interval and a viscoelastic pre-segment to the sum of the elastic interval and a viscoelastic interval, within the target tensile dynamic modulus master curve. The target compressive effective elasticity ratio is the ratio of the sum of an elastic interval and a viscoelastic pre-segment to the sum of the elastic interval and a viscoelastic interval, within the target compressive dynamic modulus master curve.

In an improvement of the above embodiment, the method for predicting the tensile-compressive fatigue characteristics of a recycled asphalt mixture further comprises: extracting a target compressive effective viscoelastic ratio from the target compressive dynamic modulus master curve, wherein the target compressive effective viscoelastic ratio is the proportion of a viscoelastic pre-segment within a viscoelastic interval in the target compressive dynamic modulus master curve; and inputting the target compressive effective viscoelastic ratio into a pre-established second viscoelastic-compressive fatigue curve to generate the target compressive fatigue characteristics.

In an improvement of the above embodiment, the method for constructing the viscoelastic-tensile fatigue curve comprises: constructing a reference tensile dynamic modulus master curve for a reference asphalt mixture; extracting a reference tensile effective elasticity ratio from the reference tensile dynamic modulus master curve; performing a direct tensile fatigue test on the reference asphalt mixture to generate reference tensile fatigue characteristics; and generating the viscoelastic-tensile fatigue curve based on the relationship between the reference tensile effective elasticity ratio and the reference tensile fatigue characteristics.

In an improvement of the above embodiment, the method for constructing the first viscoelastic-compressive fatigue curve comprises: constructing a reference compressive dynamic modulus master curve for a reference asphalt mixture; extracting a reference compressive effective elasticity ratio from the reference compressive dynamic modulus master curve; performing a direct compressive fatigue test on the reference asphalt mixture to generate reference compressive fatigue characteristics; and generating the first viscoelastic-compressive fatigue curve based on the relationship between the reference compressive effective elasticity ratio and the reference compressive fatigue characteristics.

In an improvement of the above embodiment, the method for constructing the second viscoelastic-compressive fatigue curve comprises: constructing a reference compressive dynamic modulus master curve for a reference asphalt mixture; extracting a reference compressive effective viscoelastic ratio from the reference compressive dynamic modulus master curve; performing a direct compressive fatigue test on the reference asphalt mixture to generate reference compressive fatigue characteristics; and generating the second viscoelastic-compressive fatigue curve based on the relationship between the reference compressive effective viscoelastic ratio and the reference compressive fatigue characteristics.

In an improvement of the above embodiment, the method for predicting the tensile-compressive fatigue characteristics of a recycled asphalt mixture further comprises: extracting evaluation indicators from a target dynamic modulus master curve, wherein the target dynamic modulus master curve includes the target tensile dynamic modulus master curve and the target compressive dynamic modulus master curve; and predicting the viscoelastic performance of the target asphalt mixture based on the relationships between the evaluation indicators.

In an improvement of the above embodiment, the evaluation indicators include time-based indicators, interval-based indicators, and ratio-based indicators. The time-based indicators include a stress relaxation start time, a maximum flow time, and a limit stiffness time. The interval-based indicators include a viscoelastic interval, a viscoelastic pre-segment, and a viscoelastic post-segment. The ratio-based indicators include an elasticity proportion, an effective elasticity ratio, and an effective viscoelastic ratio.

Accordingly, the present invention also provides a computer device, comprising a memory and a processor, wherein the memory stores a computer program, and the processor executes the computer program to implement the steps of the above-described method for predicting the tensile-compressive fatigue characteristics of a recycled asphalt mixture.

Accordingly, the present invention also provides a computer-readable storage medium on which a computer program is stored, wherein the computer program, when executed by a processor, implements the steps of the above-described method for predicting the tensile-compressive fatigue characteristics of a recycled asphalt mixture.

The implementation of the present invention provides the following beneficial effects:

The present invention, through pre-calculation, clarifies the potential relationship between the effective elasticity ratio and fatigue characteristics, thereby constructing specific viscoelastic-tensile and first viscoelastic-compressive fatigue curves. The tensile-compressive fatigue characteristics of a target recycled asphalt mixture can be rapidly predicted using these curves, which significantly improves the prediction accuracy of fatigue characteristics and reduces the amount and time of fatigue testing.

Simultaneously, the present invention introduces a second viscoelastic-compressive fatigue curve, allowing for the prediction of target compressive fatigue characteristics through a multi-curve approach to ensure the accuracy of the target compressive fatigue characteristics.

Furthermore, the present invention extracts evaluation indicators to construct an HRAM viscoelastic evaluation system. By proceeding from the fundamental nature of viscoelasticity, the viscoelastic performance of the target asphalt mixture is effectively predicted, thereby providing a basis for the characterization and understanding of HRAM tensile-compressive fatigue characteristics.

### BRIEF DESCRIPTION OF THE DRAWINGS

FIG. 1 is a flowchart of a first embodiment of the method for predicting tensile-compressive fatigue characteristics of a recycled asphalt mixture according to the present invention.

FIG. 2 is a schematic diagram of a dynamic modulus master curve according to the present invention.

FIG. 3 is a flowchart of a second embodiment of the method for predicting tensile-compressive fatigue characteristics of a recycled asphalt mixture according to the present invention.

FIG. 4 is a flowchart of a third embodiment of the method for predicting tensile-compressive fatigue characteristics of a recycled asphalt mixture according to the present invention.

FIG. 5 is a schematic diagram of compressive dynamic modulus master curves according to the present invention.

FIG. 6 is a schematic diagram of tensile dynamic modulus master curves according to the present invention.

FIG. 7 is a log-log plot of direct tensile fatigue life versus strain according to the present invention.

FIG. 8 is a schematic diagram showing the relationship between direct compressive fatigue life and the compressive effective elasticity ratio and compressive effective viscoelastic ratio according to the present invention.

FIG. 9 is a schematic diagram showing the relationship between direct tensile fatigue life and the tensile effective elasticity ratio according to the present invention.

### DETAILED DESCRIPTION

To make the objectives, technical solutions, and advantages of the present invention clearer, the present invention will be further described in detail below with reference to the accompanying drawings.

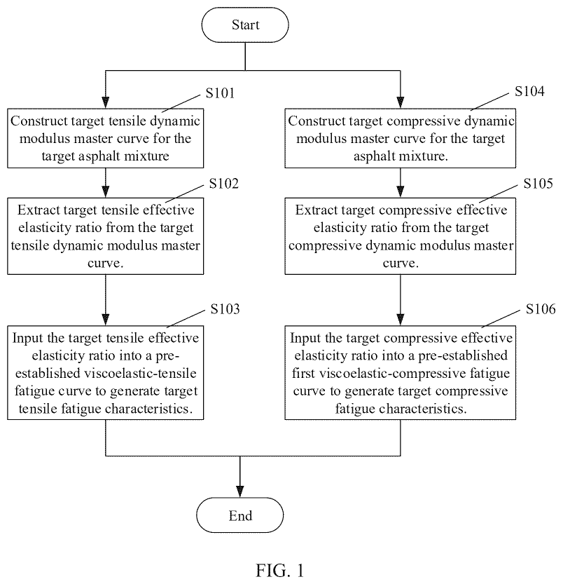

Referring to FIG. 1, a flowchart of a first embodiment of the method for predicting tensile-compressive fatigue characteristics of a recycled asphalt mixture according to the present invention is shown, which includes:

S101, constructing a target tensile dynamic modulus master curve for a target asphalt mixture.

The target tensile dynamic modulus master curve for the target asphalt mixture is constructed according to the dynamic modulus master curve construction method described in “ZHANG Jinxi, JIANG Fan, WANG Chao et al. Evaluation of dynamic modulus of indoor and outdoor aged asphalt mixtures[J]. Journal of Building Materials, 2017, 20(06): 937-942.”

S102, extracting a target tensile effective elasticity ratio from the target tensile dynamic modulus master curve.

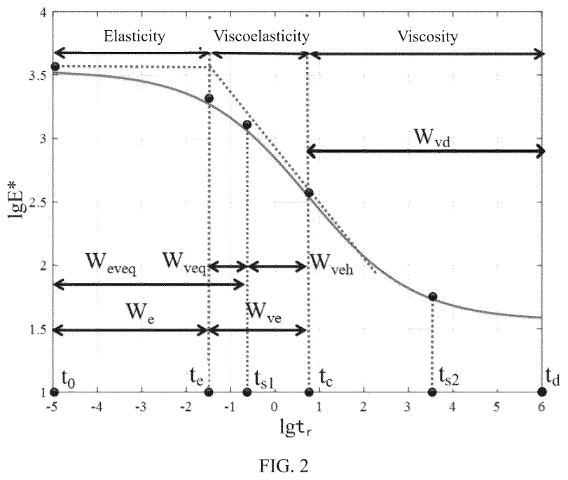

As shown in FIG. 2, E* represents the dynamic modulus, and tᵣ represents the reduced time. The dynamic modulus master curve can be divided into an elastic interval Wₑ, a viscoelastic interval Wᵥₑ, a viscous interval W_{vd}, a viscoelastic pre-segment W_{veq}, and a viscoelastic post-segment Wᵥₑₕ.

Here, the target tensile effective elasticity ratio Rₑᵥₑ is the ratio of the sum of the elastic interval Wₑ and the viscoelastic pre-segment W_{veq} to the sum of the elastic interval Wₑ and the viscoelastic interval Wᵥₑ in the target tensile dynamic modulus master curve, i.e.:

$${R}_{eve} = ( {W}_{e} + {W}_{veq} ) / ( {W}_{e} + {W}_{ve} )$$

S103, inputting the target tensile effective elasticity ratio into a pre-established viscoelastic-tensile fatigue curve to generate target tensile fatigue characteristics.

Further, the method for constructing the viscoelastic-tensile fatigue curve includes:

(1) Constructing a reference tensile dynamic modulus master curve for a reference asphalt mixture.

(2) Extracting a reference tensile effective elasticity ratio from the reference tensile dynamic modulus master curve.

(3) Performing a direct tensile fatigue test on the reference asphalt mixture to generate reference tensile fatigue characteristics.

The direct tensile fatigue test is performed on the target asphalt mixture according to the European standard EN 12697-26 Annex D test method to generate the reference tensile fatigue characteristics.

(4) Generating the viscoelastic-tensile fatigue curve based on the relationship between the reference tensile effective elasticity ratio and the reference tensile fatigue characteristics.

It should be noted that a strong relationship exists between the reference tensile effective elasticity ratio and the reference tensile fatigue characteristics at different strain levels. Therefore, the viscoelastic-tensile fatigue curve can be constructed based on this relationship.

Accordingly, when establishing the relationship between the reference tensile effective elasticity ratio and the reference tensile fatigue characteristics, it is necessary to ensure that the effective elasticity ratio and the fatigue characteristics are under the same stress state (either compression or tension) to ensure directional consistency. For example, the reference tensile effective elasticity ratio must correspond to the reference tensile fatigue characteristics, and a reference compressive effective elasticity ratio must correspond to reference compressive fatigue characteristics.

Therefore, by inputting the target tensile effective elasticity ratio into the viscoelastic-tensile fatigue curve, the target tensile fatigue characteristics of the target asphalt mixture can be generated.

S104, constructing a target compressive dynamic modulus master curve for the target asphalt mixture.

S105, extracting a target compressive effective elasticity ratio from the target compressive dynamic modulus master curve.

Here, the target compressive effective elasticity ratio is the ratio of the sum of the elastic interval and the viscoelastic pre-segment to the sum of the elastic interval and the viscoelastic interval in the target compressive dynamic modulus master curve.

S106, inputting the target compressive effective elasticity ratio into a pre-established first viscoelastic-compressive fatigue curve to generate target compressive fatigue characteristics.

Further, the method for constructing the first viscoelastic-compressive fatigue curve includes:

(1) Constructing a reference compressive dynamic modulus master curve for a reference asphalt mixture.

(2) Extracting a reference compressive effective elasticity ratio from the reference compressive dynamic modulus master curve.

(3) Performing a direct compressive fatigue test on the reference asphalt mixture to generate reference compressive fatigue characteristics.

The direct compressive fatigue test is performed on the target asphalt mixture according to the American standard AASHTO T378-17 (TP79) test method to generate the reference compressive fatigue characteristics.

To allow for comparison with the direct tensile fatigue test method, the loading waveform, frequency, and test temperature are kept consistent with the direct tensile fatigue test. A strain-controlled mode is also used, and the test is terminated when the vertical strain rate exceeds 2.0 for five consecutive cycles during the specimen's fatigue process.

(4) Generating the first viscoelastic-compressive fatigue curve based on the relationship between the reference compressive effective elasticity ratio and the reference compressive fatigue characteristics.

It should be noted that a strong relationship exists between the reference compressive effective elasticity ratio and the reference compressive fatigue characteristics. Therefore, the first viscoelastic-compressive fatigue curve can be constructed based on this relationship.

Therefore, by inputting the target compressive effective elasticity ratio into the first viscoelastic-compressive fatigue curve, the target compressive fatigue characteristics of the target asphalt mixture can be generated.

Accordingly, by combining the target tensile fatigue characteristics generated in step S103 and the target compressive fatigue characteristics generated in step S106, an accurate prediction of the tensile-compressive fatigue characteristics can be achieved.

Unlike the prior art, the present invention, through pre-calculation, clarifies the potential relationship between the effective elasticity ratio and fatigue characteristics, thereby constructing specific viscoelastic-tensile and first viscoelastic-compressive fatigue curves. These curves represent the relationship between the effective elasticity ratio and tensile and compressive fatigue, respectively. Thus, the tensile-compressive fatigue characteristics of similar recycled asphalt mixtures can be rapidly predicted using these curves without the need for extensive additional fatigue testing, greatly improving the prediction accuracy of fatigue characteristics and reducing testing volume and time.

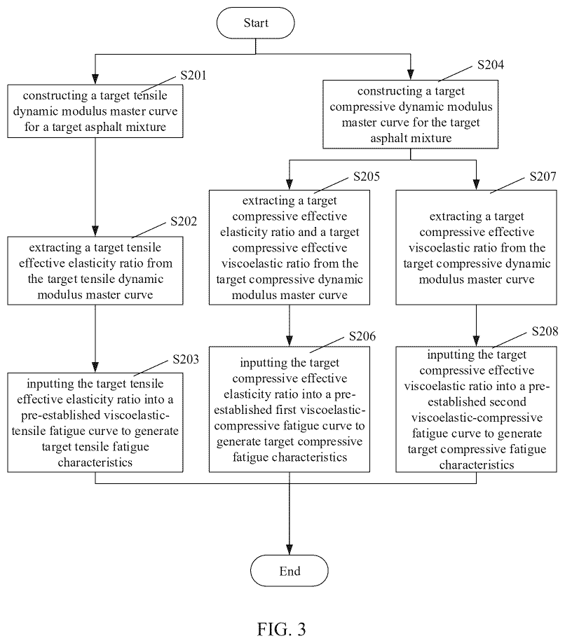

Referring to FIG. 3, a flowchart of a second embodiment of the method for predicting tensile-compressive fatigue characteristics of a recycled asphalt mixture according to the present invention is shown, which includes:

S201, constructing a target tensile dynamic modulus master curve for a target asphalt mixture.

S202, extracting a target tensile effective elasticity ratio from the target tensile dynamic modulus master curve.

S203, inputting the target tensile effective elasticity ratio into a pre-established viscoelastic-tensile fatigue curve to generate target tensile fatigue characteristics.

S204, constructing a target compressive dynamic modulus master curve for the target asphalt mixture.

S205, extracting a target compressive effective elasticity ratio and a target compressive effective viscoelastic ratio from the target compressive dynamic modulus master curve.

S206, inputting the target compressive effective elasticity ratio into a pre-established first viscoelastic-compressive fatigue curve to generate target compressive fatigue characteristics.

S207, extracting a target compressive effective viscoelastic ratio from the target compressive dynamic modulus master curve.

As shown in FIG. 2, the target compressive effective viscoelastic ratio Rᵥₑₑ is the proportion of the viscoelastic pre-segment W_{veq} within the viscoelastic interval Wᵥₑ in the target compressive dynamic modulus master curve, i.e.:

$${R}_{vee} = {W}_{veq} / {W}_{ve}$$

S208, inputting the target compressive effective viscoelastic ratio into a pre-established second viscoelastic-compressive fatigue curve to generate target compressive fatigue characteristics.

Accordingly, the method for constructing the second viscoelastic-compressive fatigue curve includes:

(1) Constructing a reference compressive dynamic modulus master curve for a reference asphalt mixture.

(2) Extracting a reference compressive effective viscoelastic ratio from the reference compressive dynamic modulus master curve.

(3) Performing a direct compressive fatigue test on the reference asphalt mixture to generate reference compressive fatigue characteristics.

(4) Generating the second viscoelastic-compressive fatigue curve based on the relationship between the reference compressive effective viscoelastic ratio and the reference compressive fatigue characteristics.

It should be noted that a strong relationship exists between the reference compressive effective viscoelastic ratio and the reference compressive fatigue characteristics. Therefore, the second viscoelastic-compressive fatigue curve can be constructed based on this relationship.

However, since the correlation between the reference compressive effective elasticity ratio and the reference compressive fatigue characteristics is higher than the correlation between the reference compressive effective viscoelastic ratio and the reference compressive fatigue characteristics, when determining the target compressive fatigue characteristics, the results from the first viscoelastic-compressive fatigue curve are considered primary, while the results from the second viscoelastic-compressive fatigue curve are considered supplementary. This multi-faceted verification of the target compressive fatigue characteristics ensures their accuracy.

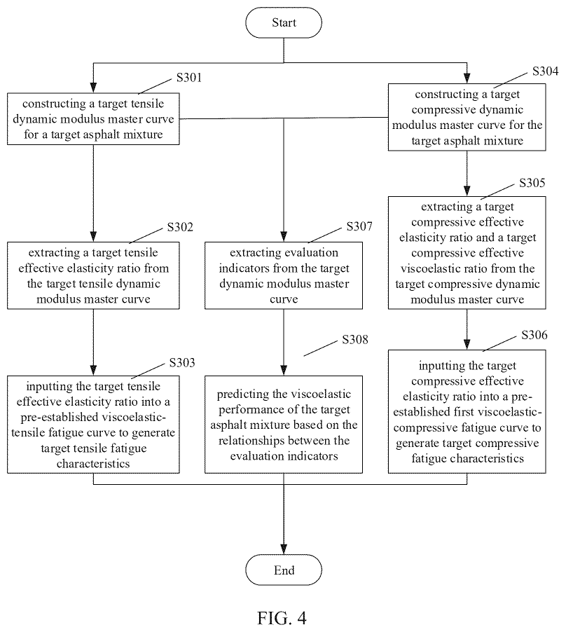

Referring to FIG. 4, a flowchart of a third embodiment of the method for predicting tensile-compressive fatigue characteristics of a recycled asphalt mixture according to the present invention is shown, which includes:

S301, constructing a target tensile dynamic modulus master curve for a target asphalt mixture.

S302, extracting a target tensile effective elasticity ratio from the target tensile dynamic modulus master curve.

S303, inputting the target tensile effective elasticity ratio into a pre-established viscoelastic-tensile fatigue curve to generate target tensile fatigue characteristics.

S304, constructing a target compressive dynamic modulus master curve for the target asphalt mixture.

S305, extracting a target compressive effective elasticity ratio and a target compressive effective viscoelastic ratio from the target compressive dynamic modulus master curve.

S306, inputting the target compressive effective elasticity ratio into a pre-established first viscoelastic-compressive fatigue curve to generate target compressive fatigue characteristics.

S307, extracting evaluation indicators from the target dynamic modulus master curve.

The target dynamic modulus master curve includes the target tensile dynamic modulus master curve and the target compressive dynamic modulus master curve. The evaluation indicators include time-based indicators, interval-based indicators, and ratio-based indicators.

As shown in FIG. 2, the dynamic modulus master curve contains rich viscoelastic information. The curve can be divided into three main intervals: elastic, viscoelastic, and viscous. By extracting key points on the dynamic modulus master curve, three categories of indicators can be formed: time-based, interval-based, and ratio-based.

### I. Time-Based Indicators

Time-based indicators include four metrics: limit elastic time t₀, stress relaxation start time tₛ₁, maximum flow time tₑ, and limit stiffness time tₑ.

Wherein:

t₀ is a fixed value of 10⁻⁵s.

tₛ₁ is the demarcation point between the elastic deformation and delayed elastic deformation of the asphalt mixture. The delayed elasticity characterizes the relaxation properties of the mixture, thus is the stress relaxation start time.

t_{c} is the inflection point where the rate of change of the asphalt mixture's stiffness transitions from fast to slow.

tₑ is the transition point where the asphalt mixture transitions from the elastic region to the viscoelastic region. Tangents are drawn to the dynamic modulus master curve at points (lg10⁻⁵, lgE*) and (lgt_{c}, lgE*), and the abscissa of the intersection of these two lines is tₑ.

### II. Interval-Based Indicators

Interval-based indicators include the elastic interval Wₑ, the viscoelastic interval Wᵥₑ, the viscous interval W_{vd}, the viscoelastic pre-segment W_{veq}, and the viscoelastic post-segment Wᵥₑₕ. Wherein,

$$[ {t}_{0} , {t}_{e} ] is {W}_{e} , [ {t}_{0} , {t}_{e} ] is {W}_{ve} , [ {t}_{c} , {t}_{d} ] is {W}_{vd} , [ {t}_{e} , {t}_{s , 1} ] is {W}_{veq} , [ {t}_{s 1} , {t}_{c} ] is {W}_{veh} , and {W}_{ve} = {W}_{veq} + {W}_{veh} .$$

### III. Ratio-Based Indicators

Ratio-based indicators include the elasticity proportion Rₑ, the effective elasticity ratio Rₑᵥₑ, and the effective viscoelastic ratio Rᵥₑₑ.

Wherein:

Rₑ represents the proportion of Wₑ in (Wₑ+Wᵥₑ), and thus 1-Rₑ is the viscoelastic proportion.

Rₑᵥₑ represents the proportion of (Wₑ+W_{veq}) in (Wₑ+Wᵥₑ).

Rᵥₑₑ represents the proportion of W_{veq} in Wᵥₑ.

The specific calculation formulas are as follows:

$$\begin{matrix}{R}_{e} = {W}_{e} / ( {W}_{e} + {W}_{ve} ) = ( {t}_{e} - {t}_{0} ) / ( {t}_{c} - {t}_{0} ) \\ {R}_{eve} = ( {W}_{e} + {W}_{veq} ) / ( {W}_{e} + {W}_{ve} ) = ( {t}_{s 1} - {t}_{0} ) / ( {t}_{c} - {t}_{0} ) \\ {R}_{vee} = {W}_{veq} / {W}_{ve} = ( {t}_{s 1} - {t}_{e} ) / ( {t}_{c} - {t}_{e} )\end{matrix}$$

Among the 12 indicators proposed above, since t₀ is a fixed value, tₑ is synonymous with Wₑ, and t_{c} is synonymous with W_{vd}, the 12 indicators are simplified to 9 indicators. The time-based indicators include the stress relaxation start time, maximum flow time, and limit stiffness time. The interval-based indicators include the viscoelastic interval, viscoelastic pre-segment, and viscoelastic post-segment. The ratio-based indicators include the elasticity proportion, effective elasticity ratio, and effective viscoelastic ratio.

S308, predicting the viscoelastic performance of the target asphalt mixture based on the relationships between the evaluation indicators.

From the definitions of each indicator, it is evident that the time-based indicators are the core of the dynamic modulus master curve, the interval-based indicators are derived from the time-based indicators, and the ratio-based indicators are derived from the interval-based indicators. The time-based indicators reflect the inherent viscoelastic properties of the asphalt mixture, while the interval-based and ratio-based indicators supplement and extend these inherent properties. By comparing these indicators for different materials, their viscoelastic performance can be distinguished.

Accordingly, the present invention also discloses a computer device, comprising a memory and a processor, wherein the memory stores a computer program, and the processor executes the computer program to implement the steps of the above-described method for predicting the tensile-compressive fatigue characteristics of a recycled asphalt mixture. Simultaneously, the present invention also discloses a computer-readable storage medium on which a computer program is stored, wherein the computer program, when executed by a processor, implements the steps of the above-described method.

Therefore, the present invention constructs specific viscoelastic-tensile and first viscoelastic-compressive fatigue curves, enabling the rapid prediction of the tensile-compressive fatigue characteristics of recycled asphalt mixtures, which greatly reduces fatigue testing volume and time. Concurrently, the invention introduces a second viscoelastic-compressive fatigue curve for multi-curve prediction of target compressive fatigue characteristics to ensure accuracy. Furthermore, the invention extracts evaluation indicators to establish an HRAM viscoelastic evaluation system, effectively predicting the viscoelastic performance of the target asphalt mixture from its fundamental nature, thereby providing a basis for the characterization and understanding of HRAM tensile-compressive fatigue properties.

The invention will now be further described in detail with reference to specific examples:

### Step (I): Reference Asphalt Mixtures

The matrix asphalt used was a commonly used 70 #grade paving petroleum asphalt with the following basic properties: penetration at 25° C. of 66.5 (0.1 mm), softening point of 47.5° C., and ductility at 10° C. of 32 cm. After thin-film oven aging, the penetration ratio at 25° C. was 69, and the ductility at 10° C. was 7.5 cm.

The aggregate was limestone from the Xinhui District of Jiangmen City, which was hot-sieved into gradations of 23-32 mm, 17-23 mm, 11-17 mm, 6-11 mm, 3.5-6 mm, and 0-3.5 mm.

The Reclaimed Asphalt Pavement (RAP) was crushed and sieved into three fractions: 0-10 mm, 10-15 mm, and 15-25 mm, with asphalt contents of 5.30%, 20.53%, and 3.30%, respectively. The basic properties of the extracted aged asphalt from the RAP were: penetration at 25° C. of 16.2 (0.1 mm), softening point of 70.6° C., and Brookfield viscosity at 135° C. of 2.95 Pa·S.

The subject of study was an AC-25 type recycled asphalt mixture with RAP contents of 0%, 30%, 45%, and 60%, abbreviated as R-0, R-30, R-45, and R-60, respectively. The R-60 mixture included an FBK-type recycling agent at a dosage of 5% by mass of the aged asphalt in the RAP, with the rejuvenator dosage included in the total asphalt content. The technical parameters of the four HRAMV groups are shown in Table 1.

TABLE 1

| | | | | | | Voids | | |
|---|---|---|---|---|---|---|---|---|
| | | | | | Voids in | Filled | | |
| | | | | | Mineral | with | | |
| | Binder | Relative Density | | Air | Aggregate | Asphalt | Stability/ | Flow/ |
| Type | Content/% | Theoretical | Measured | Voids/% | (VMA)/% | (VFA)/% | kN | mm |
| R-0 | 3.80 | 2.580 | 2.560 | 4.1 | 12.7 | 66.1 | 10.12 | 2.81 |
| R-30 | 3.80 | 2.568 | 2.538 | 3.6 | 12.6 | 69.1 | 12.33 | 2.30 |
| R-45 | 3.80 | 2.555 | 2.533 | 3.6 | 12.3 | 67.7 | 21.03 | 3.04 |
| R-60 | 3.80 | 2.548 | 2.528 | 4.5 | 12.7 | 64.6 | 16.78 | 2.99 |

### Step II: Constructing Dynamic Modulus Master Curves or the Reference Asphalt Mixtures

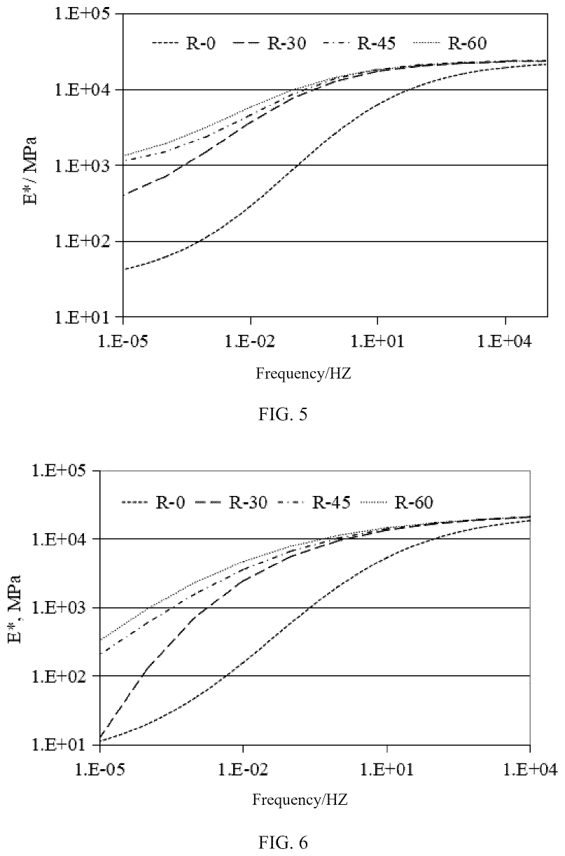

Following the dynamic modulus master curve construction method, the compressive dynamic modulus master curves (see FIG. 5) and tensile dynamic modulus master curves (see FIG. 6) for the four asphalt mixture groups were obtained. The respective parameters are shown in Table 2.

TABLE 2

| Loading | | | | | |
|---|---|---|---|---|---|
| method | Type | δ | α | β | γ |
| Compress | R-0 | 0.5897 | 2.9484 | −1.1080 | 0.6752 |
| | R-30 | 1.4418 | 2.0980 | −1.8810 | 0.7189 |
| | R-45 | 2.0902 | 1.4509 | −1.6005 | 0.7993 |
| | R-60 | 2.0716 | 1.1651 | −1.7299 | 0.7037 |
| Stretch | R-0 | −0.1115 | 3.6490 | −1.2726 | 0.6394 |
| | R-30 | −8.6119 | 12.1494 | −3.3661 | 0.4739 |
| | R-45 | −0.3971 | 3.9346 | −2.2676 | 0.4741 |
| | R-60 | −1.7241 | 5.2616 | −2.7066 | 0.4263 |

Accordingly, taking the derivative of the dynamic modulus master curve function y=f(t), it was found that the third derivative of the compressive dynamic modulus master curve has two zero points, whereas the number of zero points for the third derivative of the tensile dynamic modulus master curve is inconsistent, exhibiting cases with either two zero points or only one zero point. When the third derivative has two zero points, the values of the derivative in each interval are as shown in Table 3. When there is only one zero point, the values are as shown in Table 4.

When the third derivative of the dynamic modulus master curve has two zero points, f′(t)<0, the master curve is monotonically decreasing; f′(t_{c})=0, so t_{c} is an inflection point of concavity for the master curve; f′″(tₛ₁)=0 and f′″(tₛ₂)=0, so tₛ₁ and tₛ₂ are inflection points of the derivative function f′″(t). When there is only one zero point, as shown in Table 4, f′(t)<0, the master curve is monotonically decreasing; f″(tc)=0, so t_{c} is an inflection point of concavity for the master curve; f′″(tₛ₁)=0, so tₛ₁ is an inflection point of the derivative function f′(t).

TABLE 3

| t | (−∞, tₛ₁) | (tₛ₁, t_{c}) | (t_{c}, tₛ₂) | (tₛ₂, +∞) |
|---|---|---|---|---|
| f′(t) | − | − | − | − |
| f″(t) | − | − | + | + |
| f′″(t) | − | + | + | − |

TABLE 4

| t | (−∞, tₑ) | (tₑ, tₛ₁) | (tₛ₁, t_{c}) | (t_{c}, +∞) |
|---|---|---|---|---|
| f′(t) | − | − | − | − |
| f″(t) | − | − | − | + |
| f′″(t) | − | −+ | + | + |

Step (III): Extracting Effective Elasticity Ratio and/or Effective Viscoelastic Ratio from the Dynamic Modulus Master Curves

The values of each indicator extracted from the dynamic modulus master curves are shown in Table 5 and Table 6.

TABLE 5
Indicator Values from Compressive Dynamic Modulus Master Curves

| | Time-based Indicators | | | Interval-based Indicators | | | Ratio-based Indicators | | |
|---|---|---|---|---|---|---|---|---|---|
| Type | tₑ | t_{s 1} | t_{c} | W_{veq} | Wᵥₑₕ | Wᵥₑ | Rₑ | Rₑᵥₑ | Rᵥₑₑ |
| R-0 | −1.2915 | −0.3095 | 1.641 | 0.982 | 1.951 | 2.933 | 0.558 | 0.706 | 0.335 |
| R-30 | −0.1795 | 0.7846 | 2.6165 | 0.964 | 1.832 | 2.796 | 0.633 | 0.759 | 0.345 |
| R-45 | −0.6114 | 0.4547 | 2.0024 | 1.066 | 1.548 | 2.614 | 0.627 | 0.779 | 0.408 |
| R-60 | −0.3837 | 0.6968 | 2.4583 | 1.081 | 1.762 | 2.842 | 0.619 | 0.764 | 0.380 |

TABLE 6
Indicator Values from Tensile Dynamic Modulus Master Curves

| | Time-based Indicators | | | Interval-based Indicators | | | Ratio-based Indicators | | |
|---|---|---|---|---|---|---|---|---|---|
| Type | tₑ | t_{s 1} | t_{c} | W_{veq} | Wᵥₑₕ | Wᵥₑ | Rₑ | Rₑᵥₑ | Rᵥₑₑ |
| R-0 | −1.1677 | −0.0694 | 1.990 | 1.098 | 2.060 | 3.158 | 0.548 | 0.705 | 0.348 |
| R-30 | 2.8073 | 4.324 | 7.103 | 1.517 | 2.779 | 4.296 | 0.645 | 0.770 | 0.353 |
| R-45 | 0.5989 | 2.0052 | 4.783 | 1.406 | 2.778 | 4.184 | 0.572 | 0.716 | 0.336 |
| R-60 | 1.7103 | 3.2598 | 6.349 | 1.550 | 3.089 | 4.639 | 0.591 | 0.728 | 0.334 |

Accordingly, the compressive effective elasticity ratio Rₑᵥₑ and compressive effective viscoelastic ratio Rᵥₑₑ corresponding to the compressive dynamic modulus master curves were extracted, and the tensile effective elasticity ratio Rₑᵥₑ corresponding to the tensile dynamic modulus master curves was extracted.

Furthermore, a comprehensive analysis of the indicator values shows:

(1) In the common service time domain, the third derivatives of the compressive dynamic modulus master curves for all four mixtures have two zero points, tₛ₁ and tₛ₂. The third derivative of the tensile dynamic modulus master curve for R-0 also has both tₛ₁ and tₛ₂. However, the third derivatives for the R-30, R-45, and R-60 tensile master curves have only one zero point, tₛ₁, with no tₛ₂. In compression mode, all four asphalt mixtures exhibit elastic, viscoelastic, and viscous responses. In tension mode, R-0 exhibits elastic, viscoelastic, and viscous responses, while the three recycled asphalt mixtures R-30, R-45, and R-60 only exhibit elastic and viscoelastic responses, with no viscous response.

(2) In compression mode, compared to the virgin asphalt mixture, the time-based indicators (tₑ, tₛ₁, t_{c}) of R-30, R-45, and R-60 are shifted to the right overall. The interval-based indicator Wᵥₑ decreases for all, and the ratio-based indicator Rₑ increases for all, indicating that the elastic interval of the asphalt binder becomes larger, the elasticity proportion increases, and the viscoelastic interval becomes smaller. As the RAP content increases, Wᵥₑₕ and W_{veq} first decrease and then increase, indicating a change in the proportion of the viscoelastic pre-segment and post-segment within the viscoelastic interval. Both Rₑᵥₑ and Rᵥₑₑ increase, enhancing the effective elasticity and effective viscoelastic proportion of the asphalt mixture. In compression mode, the overall Wᵥₑ becomes smaller, but the proportions of Rₑᵥₑ and Rᵥₑₑ become larger. The change in W_{veq} is related to the stress-bearing characteristics of the asphalt mixture's material composition system. In compression mode, the asphalt mixture relies on the entire system of asphalt and aggregate to resist external loads, including cohesive forces, aggregate skeleton interlock, etc. The aged and hardened asphalt in RAP increases the cohesive force of the asphalt binder, which, coupled with the aggregate interlock, causes Wᵥₑ to decrease and the proportions of Rₑᵥₑ and Rᵥₑₑ to increase. It can be seen that in compression mode, compared to the virgin asphalt mixture, the recycled mixtures are “elastic and insufficiently viscoelastic, but sufficiently effective viscoelastic.”

(3) In tension mode, the three recycled asphalt mixtures show no viscous response. Compared to the virgin asphalt mixture, the recycled mixtures are “elastic but not viscous.” In tension mode, the mixture primarily relies on the cohesive force between the asphalt binder and the aggregate to resist external loads. Near the maximum flow time, the mixture system has sufficient strength reserve in compression mode to counteract external forces. In contrast, in tension mode, the cohesive force between the asphalt binder and the aggregate is insufficient to resist external loads. This demonstrates that different resistance mechanisms in the mixture system cause significant differences in the viscoelastic response under compressive and tensile stress modes.

(4) Based on the viscoelastic response characteristics of recycled asphalt mixtures in tension mode, a comparative analysis of the three recycled mixtures was conducted. As the RAP content increases, all eight indicators, except for Rᵥₑₑ which consistently decreases, exhibit a trend of first decreasing and then increasing. Furthermore, in tension mode, Rᵥₑₑconsistently decreases with increasing RAP content. Although this indicator shows good applicability in compression mode, its applicability in tension mode is weak.

(5) In compression mode, the time-based, interval-based, and ratio-based indicators for R-60 generally fall between those of R-30 and R-45. In tension mode, the time-based and ratio-based indicators for R-60 also generally fall between those of R-30 and R-45, indicating that the addition of the recycling agent partially restores both elasticity and viscoelasticity.

Step (IV): Performing Direct Compressive and Direct Tensile Fatigue Tests on the Reference Asphalt Mixtures The tests were performed to generate reference tensile fatigue characteristics. The specific test methods are shown in Table 7.

TABLE 7

| | Temperature / | Strain | | |
|---|---|---|---|---|
| Test Parameter | ° C. | Level / με | Waveform | Test Method |
| Direct | 20 | 100 | Haversine | AASHTO |
| Compression | | | | T378-17(TP79) |
| Direct Tension | 20 | 100, 125, | Haversine | EN12697- |
| | | 150, 175 | | 26Annex D |

It should be noted that the direct compression test is time-consuming. To enable comparison with the direct tension test, only the direct compression test at a strain level of 100 μ_{ε} was conducted. Three specimens were tested at each strain level. The direct compression test results are shown in Table 8.

TABLE 8

| | | | | Standard | |
|---|---|---|---|---|---|
| | | Fatigue Life / | Average | Deviation / | Coefficient |
| Type | No. | cycles | Life / cycles | cycles | of Variation |
| R-0 | 1 | 682120 | 673310 | 83016 | 12% |
| | 2 | 586240 | | | |
| | 3 | 751570 | | | |
| R-30 | 1 | 751230 | 896940 | 151803 | 17% |
| | 2 | 885420 | | | |
| | 3 | 1054180 | | | |
| R-45 | 1 | 1386350 | 1165877 | 194467 | 17% |
| | 2 | 1092530 | | | |
| | 3 | 1018750 | | | |
| R-60 | 1 | 925320 | 961720 | 178241 | 19% |
| | 2 | 804500 | | | |
| | 3 | 1155360 | | | |

From Table 8, the ranking of direct compressive fatigue life or the our mixtures at 100 με is: R-45>R-60 >R-30>R-0.

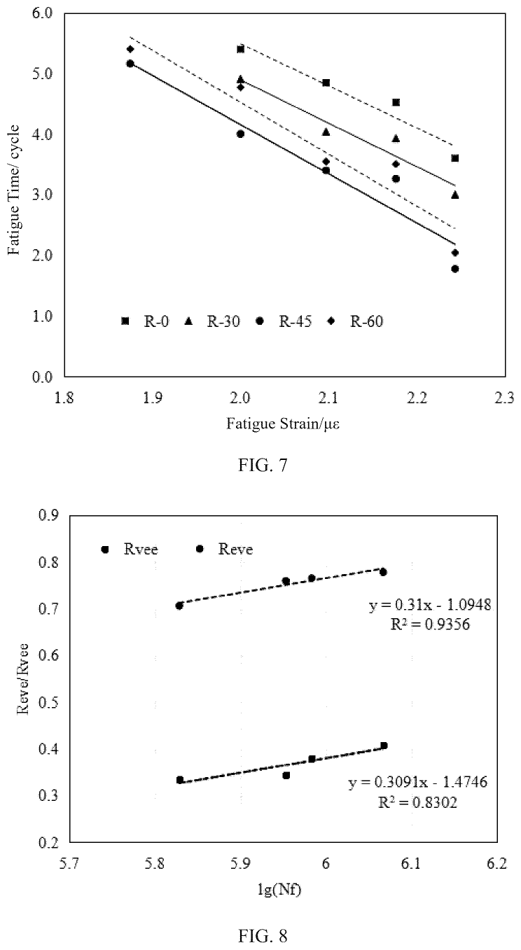

As shown in FIG. 7, in the direct tension test, the ranking of direct tensile fatigue life for the four mixtures at all strain levels is: R-0>R-30>R-60>R-45. Simultaneously, within the strain level range of this invention, the upper and lower limits of the strain level do not exceed 100 με, but the fatigue life spans two orders of magnitude, indicating that the direct tensile life is sensitive to the strain level.

Step (V): Constructing the First Viscoelastic-Compressive Fatigue Curve, the Second Viscoelastic-Compressive Fatigue Curve, and the Viscoelastic-Tensile Fatigue Curve.

Based on the relationship between the compressive effective elasticity ratio and the compressive fatigue characteristics, the first viscoelastic-compressive fatigue curve is generated: y=0.31x-1.0948 (see FIG. 8), where y is the compressive effective elasticity ratio, Nf is the compressive fatigue characteristic, and x=lg(Nf).

Based on the relationship between the compressive effective viscoelastic ratio and the compressive fatigue characteristics, the second viscoelastic-compressive fatigue curve is generated: y=0.3091x-1.4746 (see FIG. 8), where y is the compressive effective viscoelastic ratio, Nf is the compressive fatigue characteristic, and x=lg(Nf).

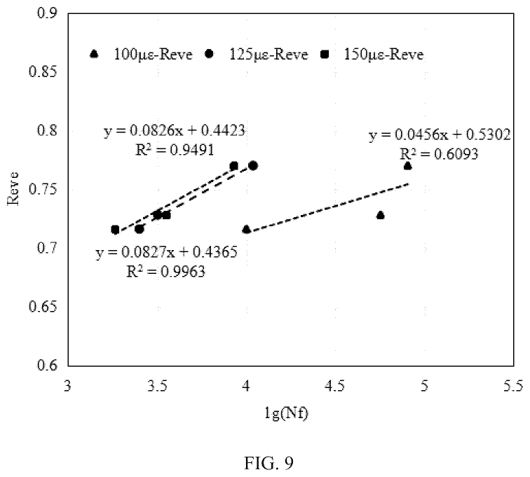

Based on the relationship between the tensile effective elasticity ratio and the tensile fatigue characteristics, the viscoelastic-tensile fatigue curves are generated: y=0.0826x+0.4423, y=0.0456x+0.5302, and y=0.0827x+0.4365, (see FIG. 9), where y is the tensile effective elasticity ratio, Nf is the tensile fatigue characteristic, and x=lg(Nf).

Step (VI): Constructing the Compressive and Tensile Dynamic Modulus Master Curves for a Target Asphalt Mixture by following the procedure of Step (II).

Step (VII): Extracting the Compressive Effective Elasticity Ratio, Compressive Effective Viscoelastic Ratio, Tensile Effective Elasticity Ratio, and Evaluation Indicators from the compressive and tensile dynamic modulus master curves by following the procedure of Step (III), and predicting the viscoelastic performance of the target asphalt mixture based on the relationships between the evaluation indicators.

Step (VIII): Calculating the Fatigue Characteristics. The compressive effective elasticity ratio is substituted into the first viscoelastic-compressive fatigue curve y=0.31x-1.0948 to calculate the compressive fatigue characteristics. The compressive effective viscoelastic ratio is substituted into the second viscoelastic-compressive fatigue curve y=0.3091x-1.4746 to verify the compressive fatigue characteristics. The tensile effective elasticity ratio is substituted into the corresponding viscoelastic-tensile fatigue curve (y=0.0826x+0.4423, y=0.0456x+0.5302, or y=0.0827x+0.4365) to calculate the tensile fatigue characteristics.

The above-described embodiments are preferred implementations of the present invention. It should be pointed out that for those of ordinary skill in the art, certain improvements and modifications can be made without departing from the principles of the present invention, and these improvements and modifications should also be considered within the scope of protection of the present invention.

1. A method for predicting tensile-compressive fatigue characteristics of a recycled asphalt mixture, the method comprising:
constructing a target tensile dynamic modulus master curve for a target asphalt mixture;
extracting a target tensile effective elasticity ratio from the target tensile dynamic modulus master curve;
inputting the target tensile effective elasticity ratio into a pre-established viscoelastic-tensile fatigue curve to generate target tensile fatigue characteristics;
constructing a target compressive dynamic modulus master curve for the target asphalt mixture;
extracting a target compressive effective elasticity ratio from the target compressive dynamic modulus master curve; and
inputting the target compressive effective elasticity ratio into a pre-established first viscoelastic-compressive fatigue curve to generate target compressive fatigue characteristics.

2. The method of claim 1, wherein:
the target tensile effective elasticity ratio is a ratio of a sum of an elastic interval and a viscoelastic pre-segment to a sum of the elastic interval and a viscoelastic interval within the target tensile dynamic modulus master curve; and
the target compressive effective elasticity ratio is a ratio of a sum of an elastic interval and a viscoelastic pre-segment to a sum of the elastic interval and a viscoelastic interval within the target compressive dynamic modulus master curve.

3. The method of claim 1, further comprising:
extracting a target compressive effective viscoelastic ratio from the target compressive dynamic modulus master curve, wherein the target compressive effective viscoelastic ratio is a proportion of a viscoelastic pre-segment within a viscoelastic interval in the target compressive dynamic modulus master curve; and
inputting the target compressive effective viscoelastic ratio into a pre-established second viscoelastic-compressive fatigue curve to generate the target compressive fatigue characteristics.

4. The method of claim 1, wherein a method for constructing the viscoelastic-tensile fatigue curve comprises:
constructing a reference tensile dynamic modulus master curve for a reference asphalt mixture;
extracting a reference tensile effective elasticity ratio from the reference tensile dynamic modulus master curve;
performing a direct tensile fatigue test on the reference asphalt mixture to generate reference tensile fatigue characteristics; and
generating the viscoelastic-tensile fatigue curve based on a relationship between the reference tensile effective elasticity ratio and the reference tensile fatigue characteristics.

5. The method of claim 1, wherein a method for constructing the first viscoelastic-compressive fatigue curve comprises:
constructing a reference compressive dynamic modulus master curve for a reference asphalt mixture;
extracting a reference compressive effective elasticity ratio from the reference compressive dynamic modulus master curve;
performing a direct compressive fatigue test on the reference asphalt mixture to generate reference compressive fatigue characteristics; and
generating the first viscoelastic-compressive fatigue curve based on a relationship between the reference compressive effective elasticity ratio and the reference compressive fatigue characteristics.

6. The method of claim 3, wherein a method for constructing the second viscoelastic-compressive fatigue curve comprises:
constructing a reference compressive dynamic modulus master curve for a reference asphalt mixture;
extracting a reference compressive effective viscoelastic ratio from the reference compressive dynamic modulus master curve;
performing a direct compressive fatigue test on the reference asphalt mixture to generate reference compressive fatigue characteristics; and
generating the second viscoelastic-compressive fatigue curve based on a relationship between the reference compressive effective viscoelastic ratio and the reference compressive fatigue characteristics.

7. The method of claim 1, further comprising:
extracting a plurality of evaluation indicators from the target dynamic modulus master curve, wherein the target dynamic modulus master curve includes the target tensile dynamic modulus master curve and the target compressive dynamic modulus master curve; and
predicting a viscoelastic performance of the target asphalt mixture based on relationships between the plurality of evaluation indicators.

8. The method of claim 7, wherein the plurality of evaluation indicators comprise time-based indicators, interval-based indicators, and ratio-based indicators;
the time-based indicators comprise a stress relaxation start time, a maximum flow time, and a limit stiffness time;
the interval-based indicators comprise a viscoelastic interval, a viscoelastic pre-segment, and a viscoelastic post-segment; and
the ratio-based indicators comprise an elasticity proportion, the effective elasticity ratio, and an effective viscoelastic ratio.

9. A computer device, comprising a memory and a processor, the memory storing a computer program, wherein the processor, when executing the computer program, is configured to perform the steps of the method of claim 1.

10. A non-transitory computer-readable storage medium on which a computer program is stored, wherein the computer program, when executed by a processor, causes the processor to perform the steps of the method of claim 1.
